(19) United States
(12) Patent Application Publication
(10) Pub. No.: US 2025/0129018 A1
(43) Pub. Date: Apr. 24, 2025

(54) PLEUROMUTILIN DERIVATIVES CONTAINING CYCLOALKYL GROUP AND PREPARATION METHODS AND APPLICATIONS THEREOF

(71) Applicant: LANZHOU INSTITUTE OF HUSBANDRY AND PHARMACEUTICAL SCIENCES OF CAAS, Lanzhou (CN)

(72) Inventors: Jianyong LI, Lanzhou (CN); Zhun LI, Lanzhou (CN); Xiwang LIU, Lanzhou (CN); Zhe QIN, Lanzhou (CN); Yajun YANG, Lanzhou (CN); Lixia BAI, Lanzhou (CN); Wenbo GE, Lanzhou (CN); Shihong LI, Lanzhou (CN); Xiao XU, Lanzhou (CN)

(21) Appl. No.: 18/883,230

(22) Filed: Sep. 12, 2024

(30) Foreign Application Priority Data
Oct. 24, 2023 (CN) 202311376279.9

(51) Int. Cl. C07C 323/52; A61K 31/221; A61P 31/04; C07C 317/44; C07C 319/12; C07C 319/28

(52) U.S. Cl. CPC C07C 323/52; A61K 31/221; A61P 31/04; C07C 317/44; C07C 319/12; C07C 319/28; C07C 2603/82

(57) ABSTRACT

Provided herein is a pleuromutilin derivative containing a cycloalkyl group and preparation method and application thereof. Specifically, the pleuromutilin is reacted with p-toluenesulfonyl chloride to obtain an intermediate I, and the intermediate I is reacted with 1-amino-2-methylpropane-2-thiol hydrochloride to obtain an intermediate II, and then the intermediate II is reacted with cycloalkylcarbonyl chloride to obtain a target derivative. The synthesis process of these derivatives is simple, with a high yield and simple purification. These derivatives can effectively inhibit activities of Staphylococcus aureus and Streptococcus, and have superior activities to the marketed Tiamulin, and they also have good antibacterial activities against drug-resistant bacteria. In particular, the treatment effect of the derivative 2 of the target derivative is significantly superior to the clinical drugs Tiamulin and Valnemulin. They are suitable as new antibacterial drugs for prevention and treatment of infectious diseases caused by bacteria in humans or animals.

### CROSS-REFERENCE TO RELATED APPLICATIONS

This application claims priority under 35 U.S.C. § 119 (a) to Chinese Patent Application No. 2023113762799, filed on Oct. 24, 2023, which is hereby incorporated by reference herein in its entirety.

### TECHNICAL FIELD

The disclosure relates to the field of medical chemistry, in particular, to a pleuromutilin derivative containing a cycloalkyl group and preparation method and application thereof.

### BACKGROUND ART

In recent years, the degree of intensification and large-scale breeding of animal husbandry in China has been greatly improved. The vigorous development of animal husbandry is also accompanied by the increasing amount of used antimicrobial drugs, leading to the increasing drug resistance of animal-derived bacteria. In addition, the animal-derived drug-resistant bacteria and their resistance can be transferred to humans through the food chain and other pathways, which also indirectly affects human life and health. In particular, Staphylococcus aureus, Streptococcus pneumoniae and the like cause millions of deaths every year, seriously threatening human health and making human patients face the situation that there would be no medicine available in medical and clinical practice. Therefore, it is particularly important to develop a new drug to combat drug resistance.

Pleuromutilin is a diterpenoid compound with antimicrobial activity isolated from the higher fungi pleurotusmutiliz (Fr.) Sacc. and pleurotus Passeckeranius Pilat, which acts on the 23SRNA of the 50S subunit of the bacterial ribosomal by inhibiting the synthesis of bacterial proteins at the ribosomal level, specifically at the binding site of the V domain of peptidyl transferase (PTC). Pleuromutilin compounds have a structure different from that of common antimicrobial mother nuclear in clinical practice, and therefore, it is not easy for the pleuromutilin compounds to induce cross-resistance with other structural antimicrobials. Although the pleuromutilin has antimicrobial activity, it is very difficult to develop it into an effective antimicrobial drug due to its slight solubility in water which results in a relatively low absorption in vivo. In contrast, appropriate derivative structural modifications made to the pleuromutilin can effectively solve this problem. Therefore, it is necessary to continuously develop more pleuromutilin derivatives with novel structures and good antimicrobial activity for subsequent clinical trials and use.

### SUMMARY

An object of the present disclosure is to provide a pleuromutilin derivative containing a cycloalkyl group and preparation method and application thereof. A specific technical scheme adopted by the present disclosure is as follows:

According to the first aspect of the present disclosure, disclosed is a pleuromutilin derivative containing a cycloalkyl group having a molecular structure represented by Formula (I):

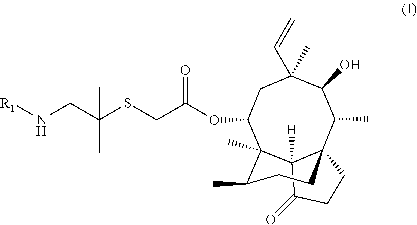

in which R₁ is one of cyclopropylcarbonyl, cyclobutylcarbonyl, cyclopentylcarbonyl, and cyclohexylcarbonyl.

According to the second aspect of the present disclosure, disclosed is a method of preparing the pleuromutilin derivative containing the cycloalkyl group, comprising the following steps:
(1) mixing pleuromutilin and p-toluenesulfonyl chloride in an alkaline solution, and adding the mixed solution into methyl tert-butyl ether under an alkaline condition of pH value greater than 13 with stirring and mixing and reacting for 1 to 3 hours to obtain a reaction product I, and sequentially subjecting the resulting reaction product I to filtration, washing and drying to obtain an intermediate 1 having a structure represented by Formula (II);

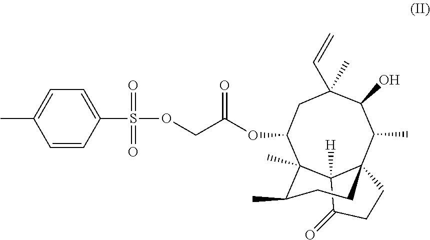

(2) adding the intermediate I and 1-amino-2-methylpropane-2-thiol hydrochloride into tetrahydrofuran and adding the alkaline solution, and when maintaining the alkaline condition of the pH value greater than 13, adding benzyltributylammonium chloride catalyst into the mixed solution and reacting for 4 to 6 hours to obtain a reaction product II, and concentrating and then sequentially subjecting the reaction product II to extraction, drying and purification to obtain an intermediate II having a structural formula represented by Formula (III);

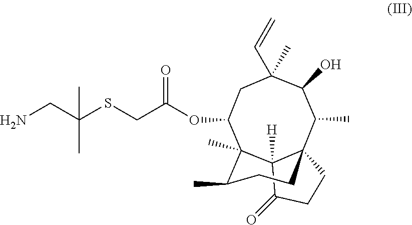 [SMILES: C=C[C@]1(C)C[C@@H](OC(=O)CSC(C)(C)CN)[C@]2(C)[C@H](C)CC[C@]3(CCC(=O)[C@H]32)[C@@H](C)[C@@H]1O]

(3) dissolving the intermediate II into dichloromethane, and then adding triethylamine and cycloalkylcarbonyl chloride, and after the addition, performing the reaction at room temperature for 2 to 6 hours to obtain a reaction product III, and then sequentially subjecting the resulting reaction product III to quenching, extraction, and drying to obtain a target derivative.

Further, the intermediate II, dichloromethane, triethylamine, and cycloalkylcarbonyl chloride in step (3) are added in a ratio of 1.08 to 1.3 mmol:5.4 to 6.48 mL:2.16 to 2.6 mmol:1.3 to 1.56 mmol.

Further, the intermediate I, 1-amino-2-methylpropane-2-thiol hydrochloride, tetrahydrofuran, benzyltributylammonium chloride, and the alkaline solution in step (2) are added in a ratio of 1.04 to 2.08 mmol:2.08 to 4.16 mL:4 to 8 mL:0.03 to 0.07 g:0.75 to 1.5 mL.

Further, the pleuromutilin, p-toluenesulfonyl chloride, methyl tert-butyl ether and the alkaline solution in step (1) are added in a ratio of 1.32 to 2.64 mmol:1.45 to 2.9 mmol:1.32 to 2.64 mL:0.22 to 0.44 mL.

Further, the alkaline solution in steps (1) and (2) is a strong alkaline solution which can be one of sodium hydroxide solution, potassium hydroxide solution, etc.

According to the third aspect of the present disclosure, disclosed is a pharmaceutically acceptable salt of the pleuromutilin derivative containing the cycloalkyl group.

Further, the pharmaceutically acceptable salt of the pleuromutilin derivative containing the cycloalkyl group is a salt formed by the derivative represented by Formula (I) with hydrochloric acid, sulfuric acid, phosphoric acid, acetic acid, fumaric acid, maleic acid, oxalic acid, propanedioic acid, glutamic acid, aspartic acid, succinic acid, citric acid, or malic acid.

According to the fourth aspect of the present disclosure, disclosed is use of the pleuromutilin derivative containing the cycloalkyl group or the pharmaceutically acceptable salt of the pleuromutilin derivative containing the cycloalkyl group in the manufacture of a medicament for treating infectious diseases.

According to the fifth aspect of the present disclosure, disclosed is an anti-bacterial drug, the drug comprising the pleuromutilin derivative containing the cycloalkyl group and at least one pharmaceutically acceptable carrier, excipient or diluent of the pleuromutilin derivative; or
the drug comprising the pharmaceutically acceptable salt of the pleuromutilin derivative containing the cycloalkyl group and at least one pharmaceutically acceptable carrier, excipient or diluent of the salt.

The technical scheme of the present disclosure has the following advantages:
(1) The present disclosure provides synthesis and extensive screening for antibacterial activity of the pleuromutilin derivative containing the cycloalkyl group. It is the first time to find that the derivative has good in vitro antibacterial activity and effect of treating bacterial infection in vivo in mice, and therefore, the derivative is particularly suitable as a new antibacterial drug for prevention and treatment of infectious diseases caused by bacteria in humans or animals. In particular, the derivative also has good antibacterial activity against Gram-positive bacteria, such as Staphylococcus aureus, streptococcus and drug-resistant bacteria (MRSA, MRSE), wherein the pleuromutilin derivative containing the cyclobutyl group has the treatment effect in mice that is superior to the marketed Tiamulin and Valnemulin.
(2) The process of preparing the pleuromutilin derivative containing the cycloalkyl group provided by the present disclosure is simple, with a high yield and simple purification process.

### BRIEF DESCRIPTION OF DRAWINGS

FIG. 1 is a nuclear magnetic resonance spectrum of derivative 2 according to one or more embodiments of the present disclosure;

FIG. 2 is a curve chart of in vivo antimicrobial activity experiment on derivative 2 in mice according to one or more embodiments of the present disclosure.

### DETAILED DESCRIPTION

For the purpose of better understanding of the object, structure and function of the present disclosure, a pleuromutilin derivative containing a cycloalkyl group of the present disclosure and preparation method and application thereof are further described in detail in combination with the accompanying drawings below.

### I. Synthesis Experiment

The method of synthesis of the pleuromutilin derivative containing the cycloalkyl group is performed according to the following reaction equation:

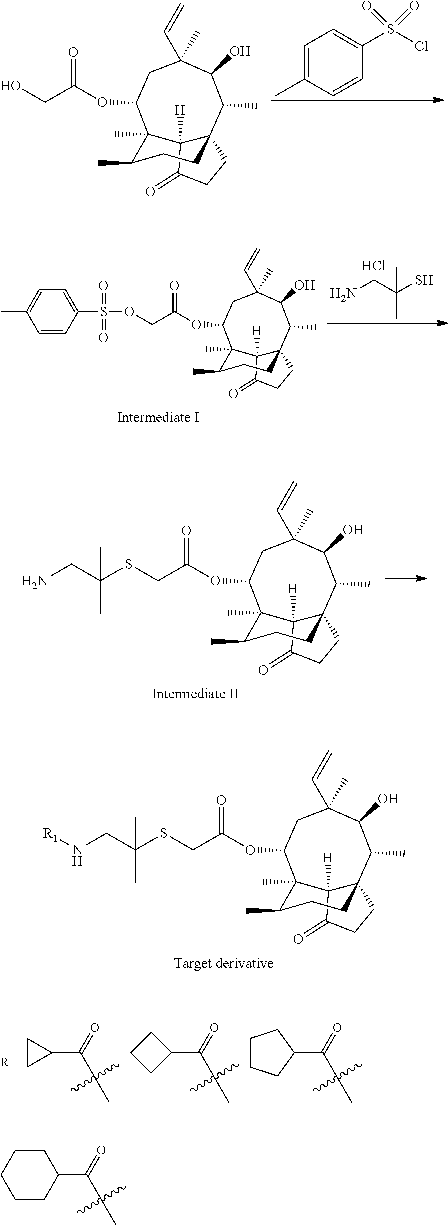

### (1) Synthesis of Intermediate I

### Example 1

1.32 mmol of the pleuromutilin and 1.45 mmol of p-toluenesulfonyl chloride were mixed, and 0.22 mL of 10 mol/L NaOH solution was added dropwise, so that the pH value of the mixed solution was greater than 13. The mixed solution was added into 1.32 mL of methyl tert-butyl ether, and then was heated to 55° C., refluxed and reacted for 1 hour. At the end of the reaction, the temperature of the mixed solution was reduced to 0° C., and a white solid obtained by filtration was sequentially rinsed with methyl tert-butyl ether for three times and deionized water for three times, and finally dried to obtain the intermediate I having a structure represented by Formula (II), with a yield of 92.9%.

### Example 2

1.32 mmol of the pleuromutilin and 2.9 mmol of p-toluenesulfonyl chloride were mixed, and 0.22 mL of 10 mol/L NaOH solution was added dropwise, so that the pH value of the mixed solution was greater than 13. The mixed solution was added into 1.32 mL of methyl tert-butyl ether, and then was heated to 55° C., refluxed and reacted for 1 hour. At the end of the reaction, the temperature of the mixed solution was reduced to 0° C., and a white solid obtained by filtration was sequentially rinsed with methyl tert-butyl ether for three times and deionized water for three times, and finally dried to obtain the intermediate I having a structure represented by Formula (II), with a yield of 89.2%.

### Example 3

2.64 mmol of the pleuromutilin and 1.45 mmol of p-toluenesulfonyl chloride were mixed, and 0.44 mL of 10 mol/L NaOH solution was added dropwise, so that the pH value of the mixed solution was greater than 13. The mixed solution was added into 1.32 mL of methyl tert-butyl ether, and then was heated to 55° C., refluxed and reacted for 1 hour. At the end of the reaction, the temperature of the mixed solution was reduced to 0° C., and a white solid obtained by filtration was sequentially rinsed with methyl tert-butyl ether for three times and deionized water for three times, and finally dried to obtain the intermediate I having a structure represented by Formula (II), with a yield of 85.9%.

### Example 4

2.64 mmol of the pleuromutilin and 1.45 mmol of p-toluenesulfonyl chloride were mixed, and 0.22 mL of 10 mol/L NaOH solution was added dropwise, so that the pH value of the mixed solution was greater than 13. The mixed solution was added into 2.64 mL of methyl tert-butyl ether, and then was heated to 55° C., refluxed and reacted for 1 hour. At the end of the reaction, the temperature of the mixed solution was reduced to 0° C., and a white solid obtained by filtration was sequentially rinsed with methyl tert-butyl ether for three times and deionized water for three times, and finally dried to obtain the intermediate I having a structure represented by Formula (II), with a yield of 84.1%.

### Example 5

2.64 mmol of the pleuromutilin and 1.45 mmol of p-toluenesulfonyl chloride were mixed, and 0.44 mL of 10 mol/L NaOH solution was added dropwise, so that the pH value of the mixed solution was greater than 13. The mixed solution was added into 2.64 mL of methyl tert-butyl ether, and then was heated to 55° C., refluxed and reacted for 1 hour. At the end of the reaction, the temperature of the mixed solution was reduced to 0° C., and a white solid obtained by filtration was sequentially rinsed with methyl tert-butyl ether for three times and deionized water for three times, and finally dried to obtain the intermediate I having a structure represented by Formula (II), with a yield of 86.6%.

### (2) Synthesis of Intermediate II

### Example 6

1.04 mmol of the intermediate I prepared from Example 1 and 2.08 mmol of 1-amino-2-methylpropane-2-thiol hydrochloride were added into 4 mL of tetrahydrofuran, and 0.75 mL of 20 wt % NaOH solution and 0.03 g of benzyltributylammonium chloride were continued to be added at 35° C., and then the temperature was increased to 50° C. followed by stirring for 6 hours. At the end of the reaction, a reaction product II was obtained, and the reaction product II was concentrated and sequentially subjected to extraction with dichloromethane, drying with anhydrous sodium sulfate, and purification with a mixed solution of petroleum ether and ethyl acetate in a volume ratio of 1:1 using column chromatography to obtain the intermediate II having a structure represented by Formula (III), with a yield of 80.1%.

### Example 7

2.08 mmol of the intermediate I prepared from Example 1 and 4.16 mmol of 1-amino-2-methylpropane-2-thiol hydrochloride were added into 8 mL of tetrahydrofuran, and 1.5 mL of 20 wt % NaOH solution and 0.07 g of benzyltributylammonium chloride were continued to be added at 35° C., and then the temperature was increased to 50° C. followed by stirring for 6 hours. At the end of the reaction, a reaction product II was obtained, and the reaction product II was concentrated and sequentially subjected to extraction with dichloromethane, drying with anhydrous sodium sulfate, and purification with a mixed solution of petroleum ether and ethyl acetate in a volume ratio of 1:1 using column chromatography to obtain the intermediate II having a structure represented by Formula (III), with a yield of 88.0%.

The corresponding characterization data of nuclear magnetic resonance (NMR) spectrum of the intermediate II prepared from Example 7 was as follows:

¹H NMR (400 MHz, CDCl₃) δ 6.47 (dd, J=17.4, 11.0 Hz, 1H), 5.74 (d, J=8.5 Hz, 1H), 5.33 (dd, J=11.0, 1.5 Hz, 1H), 5.19 (dd, J=17.4, 1.6 Hz, 1H), 3.34 (d, J=6.5 Hz, 1H), 3.12 (d, J=1.6 Hz, 2H), 2.59 (s, 2H), 2.39-2.26 (m, 1H), 2.27-2.15 (m, 2H), 2.13-2.01 (m, 2H), 1.76 (dd, J=14.4, 3.1 Hz, 1H), 1.68-1.62 (m, 2H), 1.57-1.48 (m, 4H), 1.45 (s, 3H), 1.43-1.37 (m, 1H), 1.31 (d, J=16.1 Hz, 1H), 1.23 (s, 6H), 1.16 (s, 3H), 1.10 (dd, J=13.9, 4.4 Hz, 1H), 0.86 (d, J=7.0 Hz, 3H), 0.72 (d, J=6.9 Hz, 3H). ¹³C NMR (101 MHz, CDCl₃) δ 217.30, 169.68, 139.29, 117.49, 74.86, 69.58, 58.47, 51.93, 48.80, 45.72, 44.99, 44.18, 42.05, 37.04, 36.27, 34.73, 31.51, 30.71, 29.95, 27.11, 26.59, 26.49, 25.11, 17.12, 15.19, 11.78. HRMS (ESI+): calcd for C₂₆H₄₃NO₄S [M+H]⁺, 466.3025; found, 466.3026.

### (3) Synthesis of Target Derivative

### Example 8

1.08 mmol of the intermediate II prepared from Example 7 was dissolved in 5.4 mL of dichloromethane, and 2.16 mmol of triethylamine and 1.3 mmol of cycloalkylcarbonyl chloride were added at 0° C. and reacted at room temperature for 2 hours. At the end of the reaction, the resulting reaction product III was sequentially subjected to quenching with saturated sodium bicarbonate solution, extraction with dichloromethane, and drying with anhydrous sodium sulfate to obtain the target derivative. Depending on the types of the added cycloalkylcarbonyl chloride, derivatives containing different alkyl chains were obtained, specifically derivative 1 (addition of cyclopropylcarbonyl chloride) with a yield of 75.6%, derivative 2 (addition of cyclobutylcarbonyl chloride) with a yield of 78.1%, derivative 3 (addition of cyclopentylcarbonyl chloride) with a yield of 80.7%, and derivative 4 (addition of cyclohexylcarbonyl chloride) with a yield of 81.9%.

### Example 9

1.3 mmol of the intermediate II prepared from Example 7 was dissolved in 6.48 mL of dichloromethane, and 2.6 mmol of triethylamine and 1.56 mmol of cycloalkylcarbonyl chloride were added at 0° C. and reacted at room temperature for 2 hours. At the end of the reaction, the resulting reaction product III was sequentially subjected to quenching with saturated sodium bicarbonate solution, extraction with dichloromethane, and drying with anhydrous sodium sulfate to obtain the target derivative. Depending on the types of the added cycloalkylcarbonyl chloride, derivatives containing different alkyl chains were obtained, specifically derivative 1 (addition of cyclopropylcarbonyl chloride) with a yield of 84.7%, derivative 2 (addition of cyclobutylcarbonyl chloride) with a yield of 86.0%, derivative 3 (addition of cyclopentylcarbonyl chloride) with a yield of 87.1%, and derivative 4 (addition of cyclohexylcarbonyl chloride) with a yield of 88.2%.

The corresponding characterization data of NMR spectrum of the derivative 1 prepared from Example 9 was as follows:

¹H NMR (400 MHz, CDCl₃) δ 6.62 (t, J=6.3 Hz, 1H), 6.50-6.41 (m, 1H), 5.74 (d, J=8.4 Hz, 1H), 5.31 (d, J=1.5 Hz, 1H), 5.18 (dd, J=17.5, 1.5 Hz, 1H), 3.40-3.25 (m, 2H), 3.23-3.03 (m, 3H), 2.34-2.05 (m, 5H), 1.77 (dq, J=14.5, 3.2 Hz, 1H), 1.70-1.61 (m, 2H), 1.59-1.47 (m, 2H), 1.46-1.32 (m, 6H), 1.31-1.22 (m, 7H), 1.16 (s, 3H), 1.11 (dd, J=14.1, 4.5 Hz, 1H), 1.00-0.93 (m, 2H), 0.88 (d, J=7.0 Hz, 3H), 0.79-0.67 (m, 5H). ¹³C NMR (101 MHz, CDCl₃) δ 216.95, 173.69, 170.13, 138.90, 117.27, 74.58, 69.93, 58.12, 47.55, 47.40, 45.44, 44.87, 43.97, 41.79, 36.69, 36.00, 34.44, 31.48, 30.41, 26.87, 26.35, 26.26, 24.84, 16.89, 14.89, 14.85, 11.55, 7.18, 7.15. HRMS (ESI+): calcd for C₃₀H₄₇NO₅S [M+H]⁺, 534.3238; found, 534.3248.

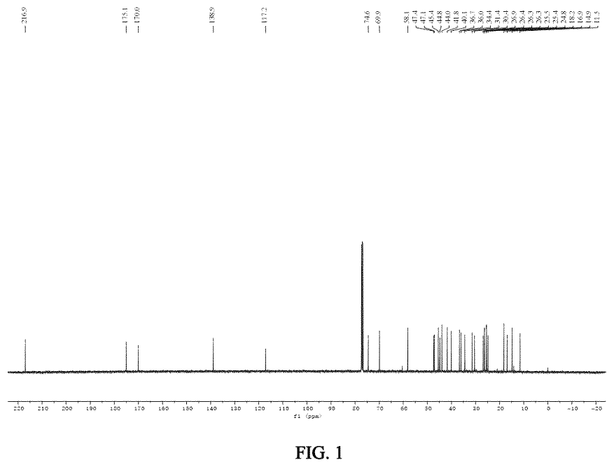

As shown in FIG. 1, the corresponding characterization data of NMR spectrum of the derivative 2 prepared from Example 9 was as follows:

¹H NMR (400 MHz, CDCl₃) δ 6.55-6.32 (m, 2H), 5.72 (dd, J=8.4, 2.1 Hz, 1H), 5.28 (d, J=10.9 Hz, 1H), 5.18 (d, J=17.4 Hz, 1H), 3.35 (d, J=6.4 Hz, 1H), 3.29-3.21 (m, 1H), 3.20-2.99 (m, 4H), 2.34-2.13 (m, 7H), 2.10-1.85 (m, 5H), 1.76 (dt, J=14.6, 2.8 Hz, 1H), 1.69-1.47 (m, 4H), 1.44 (d, J=2.2 Hz, 3H), 1.39-1.32 (m, 1H), 1.23 (dd, J=6.1, 2.0 Hz, 7H), 1.15 (d, J=2.0 Hz, 3H), 1.12-1.04 (m, 1H), 0.87 (dd, J=7.1, 1.7 Hz, 3H), 0.70 (dd, J=7.0, 2.1 Hz, 3H). ¹³C NMR (101 MHz, CDCl₃) δ 216.94, 175.08, 170.05, 138.91, 117.21, 74.56, 69.92, 58.11, 47.39, 47.09, 45.44, 44.84, 43.97, 41.78, 40.10, 36.68, 36.01, 34.44, 31.44, 30.40, 26.86, 26.37, 26.33, 26.26, 25.49, 25.43, 24.83, 18.24, 16.89, 14.87, 11.53. HRMS (ESI+): calcd for C₃₁H₄₉NO₅S [M+H]⁺, 548.3409; found, 548.3404.

The corresponding characterization data of NMR spectrum of the derivative 3 prepared from Example 9 was as follows:

¹H NMR (400 MHz, CDCl3) δ 6.54-6.36 (m, 2H), 5.72 (d, J=8.4 Hz, 1H), 5.30-5.24 (m, 1H), 5.17 (dd, J=17.4, 1.4 Hz, 1H), 3.64 (t, J=6.3 Hz, 1H), 3.44-3.20 (m, 3H), 3.17-3.10 (m, 2H), 2.62-2.52 (m, 1H), 2.35-2.27 (m, 1H), 2.24-2.15 (m, 1H), 2.11-2.02 (m, 2H), 1.89-1.85 (m, 2H), 1.80-1.72 (m, 6H), 1.66-1.62 (m, 1H), 1.59-1.53 (m, 3H), 1.46 (s, 2H), 1.43 (s, 3H), 1.36 (s, 1H), 1.30 (s, 1H), 1.25-1.20 (m, 6H), 1.15 (s, 4H), 0.87 (d, J=6.9 Hz, 3H), 0.70 (d, J=6.9 Hz, 3H). ¹³C NMR (101 MHz, CDCl3) δ 217.24, 176.54, 170.32, 139.12, 117.45, 74.77, 70.13, 58.34, 47.68, 47.36, 46.31, 45.66, 45.05, 44.18, 42.00, 36.90, 36.23, 34.68, 31.67, 30.76, 30.64, 30.62, 27.08, 26.60, 26.56, 26.49, 26.11, 26.10, 25.05, 17.14, 15.11, 11.79. HRMS (ESI+): calcd for C₃₃H₅₃NO₅S [M+H]⁺, 562.3488; found, 562.3471.

The corresponding characterization data of NMR spectrum of the derivative 4 prepared from Example 9 was as follows:

¹H NMR (400 MHz, CDCl₃) δ 6.55-6.37 (m, 2H), 5.72 (d, J=8.5 Hz, 1H), 5.29 (d, J=6.1 Hz, 1H), 5.18 (dd, J=17.4, 1.6 Hz, 1H), 3.35 (d, J=6.5 Hz, 1H), 3.28-3.07 (m, 4H), 2.35-2.28 (m, 1H), 2.26-2.16 (m, 2H), 2.15-2.03 (m, 3H), 1.92-1.86 (m, 2H), 1.81-1.73 (m, 3H), 1.70-1.60 (m, 3H), 1.53-1.41 (m, 7H), 1.38-1.29 (m, 3H), 1.27-1.19 (m, 9H), 1.15 (s, 3H), 1.10 (dd, J=14.0, 4.3 Hz, 1H), 0.87 (d, J=7.0 Hz, 3H), 0.70 (d, J=7.0 Hz, 3H). ¹³C NMR (101 MHz, CDCl₃) δ 217.18, 176.44, 170.29, 139.16, 117.46, 74.80, 70.21, 58.36, 47.69, 47.15, 45.90, 45.68, 45.08, 44.22, 42.03, 36.93, 36.26, 34.68, 31.70, 30.65, 30.03, 29.93, 27.11, 26.62, 26.60, 26.49, 26.05, 26.02, 25.07, 17.17, 15.12, 11.76. HRMS (ESI+): calcd for C₃₃H₅₃NO₅S [M+H]⁺, 576.3729; found, 576.3717.

### II. Bacteriostasis Experiment

The pleuromutilin derivatives 1-4 prepared from Example 9 of the present disclosure were determined for their effect on Methicillin-resistant Staphylococcus aureus, Methicillin-resistant Staphylococcus epidermidis, standard strains of Staphylococcus aureus (S. aureus-29213 and S. aureus-25923), clinical strains 1-48 of S. aureus, Escherichia coli, S. agalactiae and S. dysgalactiae equisimilis (S. dysgalactiae-1, S. dysgalactiae-2, S. dysgalactiae-3) and minimum inhibitory concentration (MIC) using a two-fold serial dilution method, and the results were shown in Table 1, Table 2 and Table 3. The standard strains are commercially available from ATCC, and the clinical strains are preserved by Lanzhou Institute of Husbandry and Pharmaceutical Science of Chinese Academy of Agricultural Sciences (Lanzhou, Gansu, China).

TABLE 1
The minimum inhibitory concentration
of the pleuromutilin derivatives 1-4

| | μg/mL | | | |
|---|---|---|---|---|
| | MRSA- | | | |
| Compounds | 337371 | MRSE | S. aureus-29213 | S. aureus-25923 |
| Tiamulin | 0.25 | 0.5 | 0.5 | 0.25 |
| Valnemulin | 0.031 | 0.031 | 0.063 | 0.031 |
| 1 | 0.031 | 0.031 | 0.031 | 0.031 |
| 2 | 0.031 | 0.031 | 0.063 | 0.031 |
| 3 | 0.25 | 0.25 | 0.125 | 0.125 |
| 4 | 0.125 | 0.25 | 0.125 | 0.125 |

TABLE 2
The minimum inhibitory concentration of the pleuromutilin derivatives 1-4

| | μg/mL | | | | |
|---|---|---|---|---|---|
| | E. coli- | S. agalactiae- | S. dysgalactiae- | S. dysgalactiae- | S. dysgalactiae- |
| Compounds | 25922 | 1 | 1 | 2 | 3 |
| Tiamulin | >16 | 0.25 | 0.25 | 0.25 | 0.25 |
| Valnemulin | 16 | 0.031 | 0.031 | 0.031 | 0.031 |
| 1 | >16 | 0.031 | 0.031 | 0.031 | 0.031 |
| 2 | >16 | 0.031 | 0.031 | 0.031 | 0.031 |
| 3 | >16 | 0.125 | 0.125 | 0.125 | 0.125 |
| 4 | >16 | 0.125 | 0.125 | 0.125 | 0.125 |

TABLE 3
The in vitro minimum inhibitory concentration
of the pleuromutilin derivative 2

| | Compounds | | |
|---|---|---|---|
| Strains | 2 | Tiamulin | Valnemulin |
| S. aureus-1 | 0.125 | 0.5 | 0.25 |
| S. aureus-2 | 0.063 | 0.5 | 0.25 |
| S. aureus-3 | 0.031 | 0.5 | 0.063 |
| S. aureus-4 | 0.063 | 0.5 | 0.125 |
| S. aureus-5 | 0.031 | 0.5 | 0.063 |
| S. aureus-6 | 0.031 | 0.25 | 0.063 |
| S. aureus-7 | 0.063 | 1 | 0.063 |
| S. aureus-8 | 0.063 | 0.5 | 0.063 |
| S. aureus-9 | 0.063 | 0.5 | 0.063 |
| S. aureus-10 | 0.063 | 0.5 | 0.063 |
| S. aureus-11 | 0.063 | 0.5 | 0.063 |
| S. aureus-12 | 0.031 | 0.5 | 0.031 |
| S. aureus-13 | 0.031 | 0.25 | 0.063 |
| S. aureus-14 | 0.063 | 0.5 | 0.125 |
| S. aureus-15 | 0.031 | 0.5 | 0.063 |
| S. aureus-16 | 0.031 | 0.5 | 0.063 |
| S. aureus-17 | 0.063 | 0.5 | 0.063 |
| S. aureus-18 | 0.031 | 0.125 | 0.063 |
| S. aureus-19 | 0.125 | 0.5 | 0.063 |
| S. aureus-20 | 0.063 | 0.5 | 0.125 |
| S. aureus-21 | 0.063 | 0.5 | 0.125 |
| S. aureus-22 | 0.063 | 0.5 | 0.25 |
| S. aureus-23 | 0.063 | 0.5 | 0.063 |
| S. aureus-24 | 0.063 | 0.25 | 0.063 |
| S. aureus-25 | 0.063 | 0.5 | 0.063 |
| S. aureus-26 | 0.063 | 0.5 | 0.063 |
| S. aureus-27 | 0.031 | 0.5 | 0.063 |
| S. aureus-28 | 0.063 | 1 | 0.063 |
| S. aureus-29 | 0.031 | 1 | 0.063 |
| S. aureus-30 | 0.031 | 0.5 | 0.063 |
| S. aureus-31 | 0.031 | 0.5 | 0.063 |
| S. aureus-32 | 0.063 | 1 | 0.063 |
| S. aureus-33 | 0.031 | 1 | 0.063 |
| S. aureus-34 | 0.063 | 0.5 | 0.063 |
| S. aureus-35 | 0.125 | 0.5 | 0.063 |
| S. aureus-36 | 0.016 | 0.25 | 0.063 |
| S. aureus-37 | 0.063 | 0.5 | 0.063 |
| S. aureus-38 | 0.016 | 0.5 | 0.063 |
| S. aureus-39 | 0.016 | 0.25 | 0.125 |
| S. aureus-40 | 0.016 | 1 | 0.063 |
| S. aureus-41 | 0.031 | 0.5 | 0.063 |
| S. aureus-42 | 0.063 | 0.5 | 0.063 |
| S. aureus-43 | 0.016 | 1 | 0.063 |
| S. aureus-44 | 0.031 | 0.5 | 0.063 |
| S. aureus-45 | 0.125 | 1 | 0.25 |
| S. aureus-46 | 0.063 | 1 | 0.063 |
| S. aureus-47 | 0.063 | 0.5 | 0.063 |
| S. aureus-48 | 0.063 | 0.5 | 0.125 |

It can be seen from Tables 1, 2 and 3 that pleuromutilin derivatives 1-4 have an inhibitory effect on MRSA-337371, MRSE, S. agalactiae-1, S. dysgalactiae-1, S. dysgalactiae-2, S. dysgalactiae-3, S. aureus-29213, S. aureus-25923, and clinical strains 1-48 of S. aureus (Table 3) that is superior to a control drug Tiamulin, and is superior to or equivalent to a control drug Valnemulin.

### III. In Vivo Antimicrobial Activity Experiment in Mice

### 1. Preparation and Dilution of Experimental Bacterial Solution

First, an MRSA-337371 cryopreservation solution was taken out from a freezer set at −80° C. and the following steps were performed:
(1) 10 μL of the thawed MRSA-337371 cryopreservation solution was pipetted and added into 6 mL of Tryptic Soy Broth (TSB), and incubated until the bacterial solution had become turbid;
(2) a sterile inoculation loop was dipped into and picked up a small amount of the bacterial solution and was dragged on an MH agar medium for streaking, and then the MH agar medium was inverted and placed in a constant temperature incubator at 37° C. to incubate for 24 hours;
(3) a single colony on the MH agar medium was picked and inoculated in 30 mL of MH liquid medium with an inoculation loop, and placed in a constant temperature shaking incubator at 37° C. for 18 hours for later use.

### 2. Screening of Challenge Doses

(1) 6-week-old, about 20 g-weighed, and SPF-grade healthy mice (one half male and the other female) were randomly divided into 3 groups, with 4 mice in each group (one half male and the other female), which were reared in separate cages. 100 mg/kg of cyclophosphamide was intraperitoneally injected to the mice 4 days before the experiment, and 150 mg/kg of cyclophosphamide injection was injected to the mice 1 day before the experiment to keep the mice in an immunosuppressed state for the next step.
(2) MRSA infection groups with bacterial amount of 10⁷, 10⁸ and 10⁹ CFU/mL and sterile saline control groups were provided, respectively. The route of infection was intraperitoneal injection at a dose of 0.5 mL. Each group of the experimental mice ate and drank water at their pleasure, and were continuously observed for 7 days. The number of deaths of mice was observed every day to determine minimum lethal dose MLD100, which is the challenge dose.

### 3. Experimental Grouping and Administration

6-week-old, about 20 g-weighted, SPF-grade healthy mice (one half male and the other female) were randomly divided into 5 groups, with 10 mice in each group (one half male and the other female), which were specifically divided into a positive control group (an MRSA infection group), a negative control group (a sterile normal saline control group, which was injected with only 10 mL/kg of drug solvent), drug control groups (a Tiamulin treatment group and a Valnemulin treatment group) and an experimental group (a derivative 2 treatment group). 100 mg/kg of cyclophosphamide was intraperitoneally injected to the mice 4 days before the experiment, and 150 mg/kg of cyclophosphamide injection was injected to the mice 1 day before the experiment to keep the mice in an immunosuppressed state. After fasting for 12 hours, except for the negative control group, the remaining 4 groups were intraperitoneally injected with 0.5 mL of 10⁸ CFU/mL bacterial solution, and after 30 min, the drug control groups and the experimental group were intravenously injected with 20 mg/kg of the respective drug. The mice were continued to be reared, and observed to record the deaths daily.

### 4. Observation Index

Calculation of survival rate: During the experiment, the morbidity and the mortality in each group of mice was observed and recorded in detail every day, and the survival rate in each group of mice was calculated as:

Survival rate=Number of surviving animals/Number of experimental animals×100%

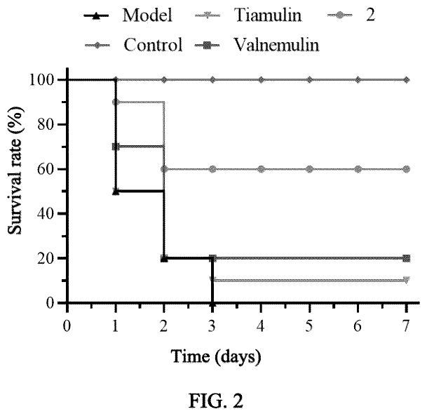

FIG. 2 shows a curve chart representing a comparison of the survival rate of mice infected with MRSA-337371 after treatment with each group of drugs.

### 5. Analysis of Experimental Results

As can be seen from FIG. 2, on day 1, the mortality rate of the mice in the positive control group (i.e., Model group) and Tiamulin group was 50%, respectively, and the mortality rate of the Valnemulin group was 30%, and the mortality rate of the derivative 2 (i.e., the pleuromutilin derivative containing the cyclobutyl group) experimental group was 10%. On day 2, the mortality rate of the positive control group, Tiamulin and Valnemulin group was 80%, respectively, and the mortality rate of the derivative 2 experimental group was 40%. On day 3, the mortality rate of the positive control group was 100%, and the mortality rate of Tiamulin group was 90%. There were no more deaths in the following 5 days. The final survival rate was 0% for the positive control group, 10% for the Tiamulin group, 20% for the Valnemulin group, 60% for the derivative 2 experimental group, and 100% for the negative control group (i.e., Control group). After comparison, it was found that the protection rate of 20 mg/kg of the derivative 2 against systemic MRSA infection in mice was 60%, which was superior to the protection rate of 10% for 20 mg/kg of Tiamulin and the protection rate of 20% for 20 mg/kg of Valnemulin.

It is understood that the present disclosure is described through some embodiments, and those skilled in the art know that various changes or equivalent substitutions may be made to these features and embodiments without departing from the spirit and scope of the present disclosure. In addition, based on the teachings of the present disclosure, modifications can be made to these features and embodiments to adapt to specific conditions and materials without departing from the spirit and scope of the present disclosure. Therefore, the present disclosure is not limited by the specific embodiments disclosed herein, and all embodiments that fall within the scope of the claims of the present application fall within the scope of protection of the present disclosure.

What is claimed is:

1. A pleuromutilin derivative containing a cycloalkyl group or a pharmaceutically acceptable salt thereof, wherein the pleuromutilin derivative containing the cycloalkyl group has a molecular structure represented by Formula (I): 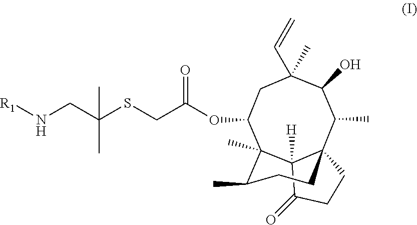
in which R₁ is one of cyclopropylcarbonyl, cyclobutylcarbonyl, cyclopentylcarbonyl, and cyclohexylcarbonyl.

2. A method for preparing the pleuromutilin derivative containing the cycloalkyl group of claim 1, wherein the method comprises the following steps:
(1) mixing pleuromutilin and p-toluenesulfonyl chloride in an alkaline solution, and adding the mixed solution into methyl tert-butyl ether under an alkaline condition of pH value greater than 13 with stirring and mixing and reacting for 1 to 3 hours to obtain a reaction product I, and sequentially subjecting the resulting reaction product I to filtration, washing and drying to obtain an intermediate 1 having a structure represented by Formula (II); 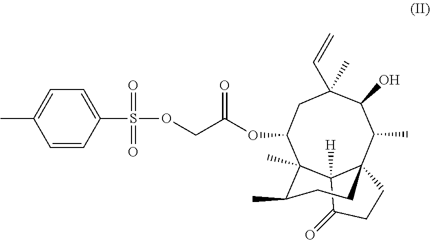
(2) adding the intermediate I and 1-amino-2-methylpropane-2-thiol hydrochloride into tetrahydrofuran and adding the alkaline solution, and when maintaining the alkaline condition of the pH value greater than 13, adding benzyltributylammonium chloride catalyst to the mixed solution and reacting for 4 to 6 hours to obtain a reaction product II, and concentrating and then sequentially subjecting the reaction product II to extraction, drying and purification to obtain an intermediate II having a structural represented by Formula (III); 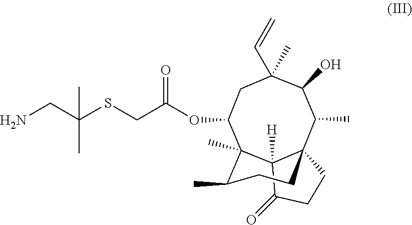 [SMILES: C=C[C@]1(C)C[C@@H](OC(=O)CSC(C)(C)CN)[C@]2(C)[C@H](C)CC[C@]3(CCC(=O)[C@H]32)[C@@H](C)[C@@H]1O]
(3) dissolving the intermediate II into dichloromethane, and then adding triethylamine and cycloalkylcarbonyl chloride, and after the addition, performing the reaction at room temperature for 2 to 6 hours to obtain a reaction product III, and then sequentially subjecting the resulting reaction product III to quenching, extraction, and drying to obtain a target derivative.

3. The method of claim 2, wherein the intermediate II, dichloromethane, triethylamine, and cycloalkylcarbonyl chloride in step (3) are added in a ratio of 1.08 to 1.3 mmol:5.4 to 6.48 mL:2.16 to 2.6 mmol:1.3 to 1.56 mmol.

4. The method of claim 2, wherein the intermediate I, 1-amino-2-methylpropane-2-thiol hydrochloride, tetrahydrofuran, benzyltributylammonium chloride, and the alkaline solution in step (2) are added in a ratio of 1.04 to 2.08 mmol:2.08 to 4.16 mL:4 to 8 mL:0.03 to 0.07 g:0.75 to 1.5 mL.

5. The method of claim 2, wherein the pleuromutilin, p-toluenesulfonyl chloride, methyl tert-butyl ether and the alkaline solution in step (1) are added in a ratio of 1.32 to 2.64 mmol:1.45 to 2.9 mmol:1.32 to 2.64 mL:0.22 to 0.44 mL.

6. The pharmaceutically acceptable salt of the pleuromutilin derivative containing the cycloalkyl group of claim 1, wherein the salt is a salt formed by the derivative represented by Formula (I) with hydrochloric acid, sulfuric acid, phosphoric acid, acetic acid, fumaric acid, maleic acid, oxalic acid, propanedioic acid, glutamic acid, aspartic acid, succinic acid, citric acid, or malic acid.

7. Use of a pleuromutilin derivative containing a cycloalkyl group or a pharmaceutically acceptable salt of claim 1 in the manufacture of a medicament for treating infectious diseases.

8. An anti-bacterial drug, wherein the drug comprises a pleuromutilin derivative containing a cycloalkyl group of claim 1 and at least one pharmaceutically acceptable carrier, excipient or diluent of the pleuromutilin derivative; or
wherein the drug comprises a pharmaceutically acceptable salt of a pleuromutilin derivative containing a cycloalkyl group of claim 1 and at least one pharmaceutically acceptable carrier, excipient or diluent of the salt.
